(19) United States
(12) Patent Application Publication
(10) Pub. No.: US 2021/0146296 A1
(43) Pub. Date: May 20, 2021

(54) METHOD FOR OPERATING A REACTOR FACILITY

(71) Applicant: Siemens Aktiengesellschaft, Munich (DE)

(72) Inventors: Manfred Baldauf, Erlangen (DE); Lukas Glöser, Erlangen (DE); Katharina Meltzer, Erlangen (DE); Alexander Tremel, Möhrendorf (DE)

(73) Assignee: Siemens Aktiengesellschaft, Munich (DE)

(21) Appl. No.: 17/047,853

(22) PCT Filed: Apr. 8, 2019
(86) PCT No.: PCT/EP2019/058797

§ 371 Date: Oct. 15, 2020

(30) Foreign Application Priority Data
Apr. 20, 2018 (EP) 18168534.8

(51) Int. Cl. B01D 53/14; B01D 53/00; B01J 12/00

(52) U.S. Cl. CPC B01D 53/1418; B01D 53/1425; B01D 2252/30; B01J 12/00; B01D 53/002

(57) ABSTRACT

A method for operating a reactor facility for equilibrium-limited reactions, includes: converting starting materials to a product in a reaction chamber under a pressure p1, wherein an absorbent is loaded with the product and absorbs starting materials; discharging the loaded absorbent from the reaction chamber; lowering the pressure of the absorbent to a pressure p2 which is lower than pressure p1 and the product and starting materials are discharged in the gaseous state from the liquid absorbent; separating the gaseous products by condensation from the gaseous starting materials at the same time as a pressure p3 higher than pressure p1 is applied to the liquid absorbent, under pressure p3 into a liquid jet gas compressor in which the gaseous starting materials separated from the products are aspirated and dissolved in the liquid absorbent; and then introduced under pressure p4, which is lower than pressure p3, into the reaction chamber.

### CROSS REFERENCE TO RELATED APPLICATIONS

This application is the US National Stage of International Application No. PCT/EP2019/058797 filed 8 Apr. 2019, and claims the benefit thereof. The International Application claims the benefit of European Application No. EP18168534 filed 20 Apr. 2018. All of the applications are incorporated by reference herein in their entirety.

### FIELD OF INVENTION

The invention relates to a method for operating a reactor system.

### BACKGROUND OF INVENTION

Fossil fuels cause carbon dioxide emissions, which make it difficult to meet global climate protection goals. For this reason, the development of renewable energies is being promoted. However, the production of regenerative power is subject to significant regional as well as temporal variations.

For example, on sunny or windy days, power is produced cost-effectively by photovoltaics or wind power plants, respectively. Economical approaches are currently being sought for putting this power to meaningful use via the electricity sector and, for example, for generating useful chemical products therefrom. One possibility is the electrochemical conversion of water into hydrogen and oxygen. The hydrogen that is produced can then react with climate-damaging carbon dioxide as starter molecule or starting material, whereby carbon dioxide emission would be reduced at the same time. Carbon dioxide, which is relatively easily available, can thus react as an inexpensive carbon source, for example in the production of methanol as a possible product of a one-stage synthesis of carbon dioxide and hydrogen according to the reaction equation

CO₂+3H₂→CH₃OH+H₂O

A disadvantage of the synthesis of methanol from carbon dioxide and hydrogen is the low equilibrium conversions, which are approximately only 20% at 50 bar and 250° C. Therefore, in conventional synthesis systems, a large part of the gaseous starting materials is circulated, which leads to considerable pressure losses in the reactor and in pipelines and whereby a considerable energy input in the form of compression power and heat power must take place. Furthermore, gas recycling is not very suitable for dynamic operation of a reaction system and can therefore also be poorly adapted to irregularly occurring amounts of electrical energy, as predominate as a result of the fluctuating production on the basis of wind and photovoltaics.

An approach for carrying out equilibrium-limited reactions has already been described in WO 2017212016 A1. There is thereby used a stirred tank reactor in which an absorbent is present in a lower region of the reactor and starting material gases are conducted through a catalyst arrangement, wherein products are absorbed by absorbers far apart from the catalyst. Such a system is wholly useful but is technically complex owing to the stirrer arrangement and has limitations in the dynamic removal of the product and the dynamic supply of electrical energy.

In the process management of reactors for carrying out equilibrium-limited reactions of the described type, it is frequently unavoidable for process-related reasons that starting materials are also absorbed by the absorbent up to the particular degree of saturation. Although this involves only a small percentage of the total input of starting materials, which lies below 10%, generally below 5%, these starting materials are lost on discharge of the absorbent and separation from the products. The economy of the process as a whole is thereby impaired.

### SUMMARY OF INVENTION

The object of the invention is to provide a method for operating a reactor system for carrying out equilibrium-limited reactions which has increased economy compared with the method from the prior art.

The object is achieved by a method for operating a reactor system having the features described herein.

The method according to the invention for operating a reactor system for carrying out equilibrium-limited reactions comprises the following steps:

First of all, starting materials (or a starting material) are converted into one (or more) products in a reaction chamber. A pressure p1 thereby prevails in the system. Since the reaction is equilibrium-limited, which means that the equilibrium does not lie wholly on the side of the products, an absorbent is used which absorbs or is laden with the products that have already formed and is discharged from the reaction chamber. When the absorbent is laden with the products, starting materials are also absorbed by the absorbent in an undesirable manner. In a second step, the absorbent, which contains substantially the products but is also laden with a small amount of starting materials, is discharged from the reaction chamber. In a further method step, the pressure of the absorbent is lowered starting from the pressure p1 to a pressure p2. The pressure p2 is thus lower than the pressure p1. When the pressure is lowered, the absorbent is freed of the products and of the starting materials, which are then both present in gaseous form. The absorbent continues to be present in liquid form. In a sub-step, the gaseous products are separated from gaseous starting materials by condensation. Both the products and the starting materials can thereby consist of only one chemical component or of multiple chemical components. This is dependent on the chosen process management and on the reaction itself.

In another sub-step of the method, a pressure p3 is applied to the liquid absorbent, wherein the pressure p3 is higher than the pressure p1 prevailing in the reaction chamber. The liquid absorbent to which the pressure p3 is applied is conducted into a liquid-jet gas compressor, in which the starting materials, which are already separated from the products and are present in gaseous form, are aspirated and dissolved in the liquid absorbent. The liquid absorbent, which is again laden with starting materials, is introduced into the reaction chamber.

Because an absorbent that is already laden with the starting materials is introduced into the reaction chamber, a certain degree of saturation of the absorbent for the absorption of starting materials prevails. This in turn means that, with ideal process management, no further starting materials can pass into the absorbent during the reaction. It would in principle also be possible to compress the starting materials separated from the products and present in gaseous form and introduce them into the reaction chamber again under the necessary reaction pressure p1. However, it is more advantageous in terms of energy and less complex in terms of the process to bring the absorbent, which must in any case be increased to that pressure p1, to the level of a pressure p3 which is slightly higher than the pressure p1 and to aspirate the gaseous starting materials and accordingly dissolve them in the absorbent liquid under the pressure p3. This is because less energy must be applied in order to apply pressure to a liquid than is necessary for applying pressure to gases, since gases are highly compressible, which is not the case with liquids.

The pressure p4 of the liquid absorbent with the starting material dissolved therein is lower than the pressure p3 but advantageously higher than or equal to the pressure p1, so that the absorbent can be introduced into the reaction chamber again without the application of further pressure or without further technical measures.

The advantage of the described method consists in introducing the starting materials into the absorbent again with a relatively low outlay in terms of energy and loading the absorbent with the starting materials so that saturation is present and no further starting materials can be absorbed during the subsequent process, and thus no further starting materials are lost from the process. The starting materials with which the absorbent is already laden are advantageously present therein in a saturated form and they are supplied to the absorbent again up to a degree of saturation in an energy-efficient process step after discharge.

In a further embodiment of the invention, the pressure of the absorbent is lowered from the pressure p1 to the pressure p2 after it has left the reaction chamber so that there is a further pressure stage, which is denoted p11, between the pressures p1 and p2. Method steps C, D and E are thereby likewise carried out both for the pressure stage p11 and for the absorbent under the pressure p2. In other words, at the intermediate pressure stage p11 too, the portion of the products discharged from the absorbent by the pressure reduction is first separated from the gaseous starting materials. Carrying out method step E analogously thereby means that the absorbent has a pressure which is higher than p11 but does not necessarily have to be higher than p1.

In principle it is of course advantageous to insert further cascaded pressure stages between the pressure stage p1 and p2 in addition to the pressure stage p11. This has the advantage that, starting from p2, the pressure of the absorbent can be increased gradually to the pressure p4 again, that is to say in multiple process steps, which has the result that the pressure difference which the particular liquid-jet gas compressor must bridge is smaller than if there was an only single-stage increase between p2 and p4. This in turn means that the liquid-jet gas compressor can be designed to be less demanding in terms of construction and thus also less cost-intensive.

The pressure p1 which prevails in the reaction chamber and under which the absorbent leaves the reaction chamber lies between 20 bar and 250 bar, depending on the reaction to be carried out and the desired yield and possible energy input. For a methanol synthesis from the starting materials carbon dioxide and hydrogen, pressures of, for example, between 50 bar and 85 bar in the reaction chamber are advantageous as the pressure p1. This pressure p1 is lowered to the pressure p2, optionally in a cascaded manner, wherein the pressure p2 lies between 1 bar and 50 bar, in principle the best discharge of products and also undesirable starting materials from the absorbent took place at pressures close to atmospheric pressure, that is to say between 1 bar and 3 bar.

It is further advantageous if the pressure difference between the pressures p1 and p11 and also between further intermediate stages up to the pressure p2 in each case lies in a range between 3 bar and 10 bar, which in turn has the result that the load on the liquid-jet gas compressor can be kept low. The pumps which compress the absorbent from one pressure stage to the next can also be designed with a correspondingly lower technical outlay.

Further embodiments and further features of the invention will be explained in greater detail by means of the following figures. The embodiments are purely schematic and exemplary and do not constitute a limitation of the scope of protection.

### BRIEF DESCRIPTION OF THE DRAWINGS

In the figures

FIG. 1 shows a reactor system with one-stage pressure relief,

FIG. 2 shows a reactor system with multi-stage pressure relief, and

FIG. 3 shows a cross-section through a liquid-jet gas compressor.

### DETAILED DESCRIPTION OF INVENTION

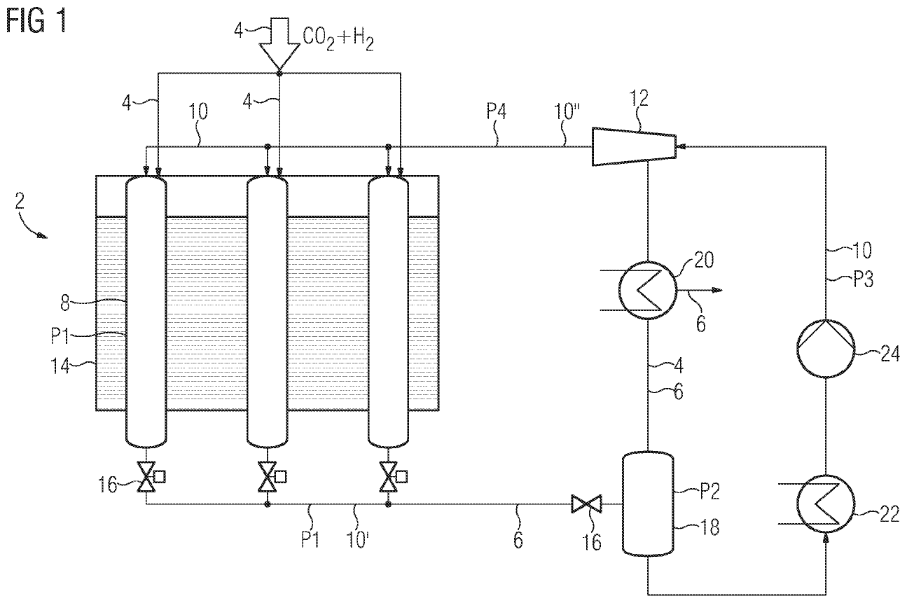

FIG. 1 first shows schematically a reactor system 2 which can be operated, for example, on the basis of a reactor bundle 14. A possible mode of functioning of such a reactor system, in particular for carrying out equilibrium-limited reactions, will be discussed briefly hereinbelow. Equilibrium-limited reactions, for example the reaction of carbon dioxide and hydrogen to methanol according to the equation

CO₂+3H₂→CH₃OH+H₂O

have the property, determined by the system because of the energy balance, that the chemical equilibrium lies more on the left side than on the right side of the reaction equation. In the described equation, the equilibrium lies approximately 20% on the right side and 80% on the left side. This has the result that, for a successful implementation of the described equation, the resulting product, or the products methanol and water, should if possible be removed from the equilibrium between the starting materials and the products. By removing the products, the prevailing equilibrium is disturbed, with the result that the system strives to achieve the equilibrium state again and to that end products are formed.

The products are advantageously transported away by an absorbent, for example by an ionic liquid. This absorbent, continuously in through-flow by suitable process management, is also continuously laden with the products formed and discharged from the reaction zone. The absorbent 10 in the reactor system 2 thereby has the prevailing process pressure p1, wherein in the described reaction an advantageous process pressure p1 is about 80 bar. However, in addition to the products methanol and water already described, traces of starting materials, that is to say of carbon dioxide and hydrogen, also dissolve in an undesirable manner in the absorbent 10. Both are useful substances in pure form. Although the solution of the starting materials in the absorbent lies only between 2% and 5%, depending on the process management in the continuous range, this portion of the cost-intensive starting materials introduced is lost if they are not fed back to the process again. Therefore, in the further described method, a possibility is provided of feeding these starting materials 4 undesirably dissolved in the absorbent 10 to the reactor system again with a comparatively low energy outlay, and thus of recycling them.

The absorbent 10 laden as described is discharged from the reaction chamber 8 via a valve 16. If, as shown in FIG. 1, there is a reactor bundle 14, the laden absorbent 10 is discharged from each individual reaction chamber 8 of the reactor bundle 14 via a corresponding valve 16 and optionally guided via a common line to a pressure chamber 18, wherein further valves 16 can be arranged in that path. In the pressure chamber 18, the pressure p1 of the absorbent, which in the laden state is provided with the reference numeral 10′, is relieved, that is to say the pressure p1 is lowered to a pressure p2. Depending on the load state and the process management, the pressure p2 is significantly lower than the pressure p1. If the pressure p1 lies in the region of approximately 80 bar, then the pressure p2 is closer to atmospheric pressure between 1 bar and 5 bar, generally not more than 10 bar. As a result of the pressure relief of the absorbent 10′, it loses the ability to absorb products and starting materials, which thereby dissolve out of the absorbent 10′ and escape in gaseous form. The absorbent 10 so unloaded is optionally cooled in a heat exchange device or cooling device 22, and pressure is applied thereto again by a pump 24. This pressure is denoted p3, wherein p3 is higher than p2 and, as will be explained, should be higher than p1 in this embodiment. For the pressure p3, a pressure that lies in the region of 100 bar is optionally to be chosen.

In a parallel process step, the product 6 which has escaped in the form of a gas, and which is present in gaseous form still mixed with the starting material 4 also discharged from the absorbent 10, is cooled in a condensation device 20, wherein the substances water and methanol, which in this case constitute the products 6, condense in the cooling device. The starting materials 4, that is to say the carbon dioxide and the hydrogen, do not condense in conventional condensation devices and remain in the gaseous phase. This is a suitable separating method for separating the undesired starting materials present and the products 6 of value.

In the absence of the attempt to make the production process as cost-effective as possible, the starting materials 4 could then be discharged into the environment, but it would also be possible to compress them by means of a compressor to such an extent that they could be fed under the process pressure p1 to the reaction chamber 8 again. However, since the starting materials 4 separated from the condensation device 20 are gases, a very large amount of energy would have to be introduced in order to compress these gases to the pressure of about 80 bar, that is to say the pressure p1, again. The outlay in terms of energy which would be required therefor would correspond approximately, depending on the price level of the raw materials, to the value which the raw materials in any case already have, so that it would also be similarly economical or uneconomical to freely release the starting materials 4 from the condensation device 20 into the environment.

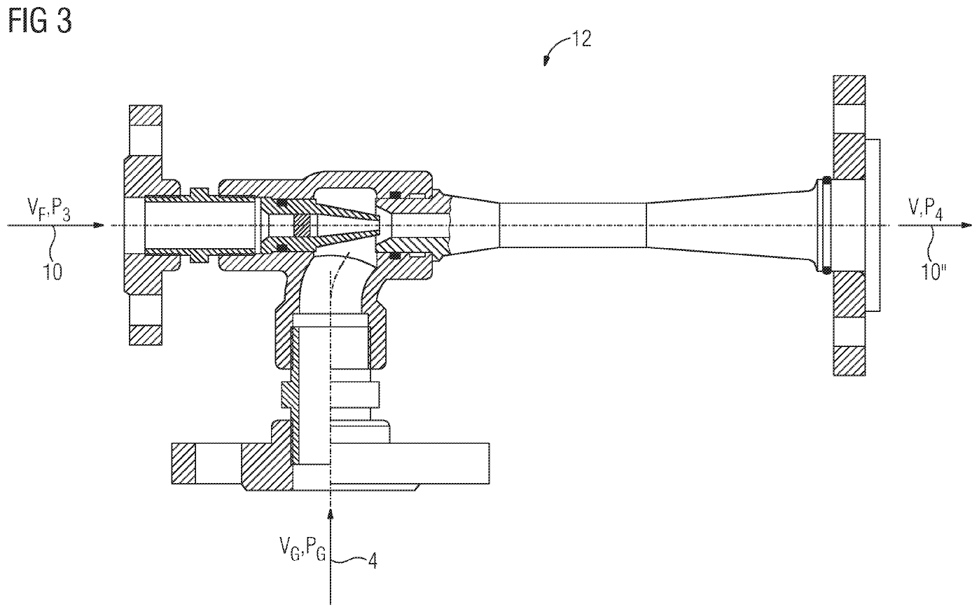

It is provided in the present method to supply a so-called liquid-jet gas compressor 12 to the absorbent 10, which now has the pressure p3. Such a liquid-jet gas compressor functions similarly to a water-jet pump in the form that a liquid is conducted at high pressure through a nozzle and thereby aspirates a gas via a further supply line. A liquid-jet gas compressor 12 is shown schematically in FIG. 3. This gas compressor has substantially three openings, one of the openings is denoted V_{F}, which stands for a volume flow rate V of the absorbent 10. The absorbent 10 has a pressure p3 on entering the gas compressor 12 and a pressure p4 at the exit, which is shown on the right-hand side. At the exit, the absorbent 10 has the reference numeral 10″, the volume flow rate is denoted V at this point. The pressure p4 is lower than the pressure p3. Through a third opening, gas with the volume flow rate V_{G} (G for gas) is introduced under a pressure pG into the gas compressor 12. This gas contains or consists substantially of the separated starting materials 4. The pressure pG is not substantially higher than atmospheric pressure, which has the result that the pressure p3 is reduced to the pressure p4 when the gas in the form of the starting materials 4 is mixed with the absorbent 10 in the gas compressor 12.

The pressure p4 is then, as already mentioned, correspondingly lower than the pressure p3, wherein the system is correspondingly so designed that the pressure p4 is close to the pressure p1, so that the absorbent 10″ laden with the starting materials can if possible be fed into the reaction chamber 8 under the pressure p1 again without a complex pressure correction.

The advantage of the described method is that the discharged starting material 4 or the starting materials 4 are present in gaseous form, and under the pressure pG, which lies close to atmospheric pressure, do not require substantial working up and can be mixed with the absorbent 10 again. An appreciable energy input for the starting material gas 4 is thus not necessary. Only an energy input for increasing the absorbent 10 to a pressure p3, which is slightly higher than the process pressure p1, is required. The additional energy outlay accordingly consists merely in applying the pressure difference Δp between p3 and p4 (Δp=p3−p4). Since the absorbent 10 is a liquid, the energy input for a pressure difference between, for example, 80 bar and 90 bar or 100 bar is comparatively small. The energy input into the absorbent 10 for producing Δp is at least significantly smaller than the energy input which would be necessary to increase the starting material 4 in gaseous form from atmospheric pressure to the pressure p1. This saved energy outlay is ultimately the contribution which allows the system to become more economical compared with the prior art.

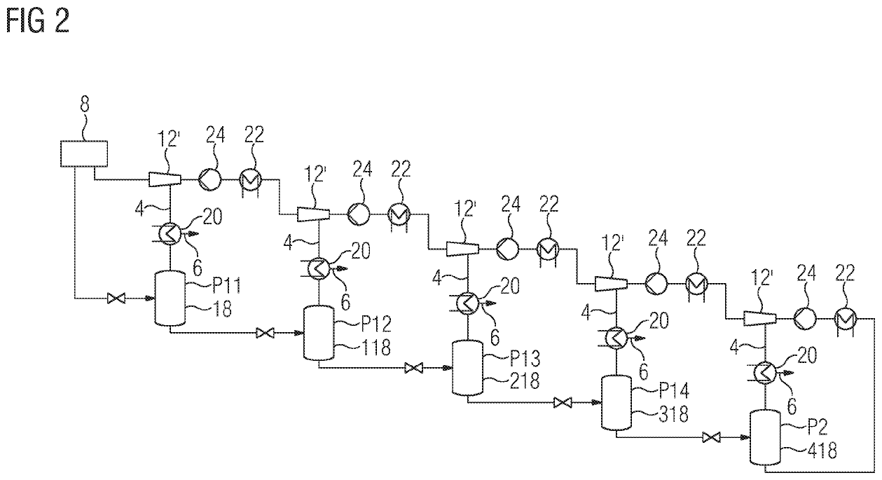

If the pressure difference between p3 and p4 in the liquid-jet gas compressor 12 were to be reduced, the gas compressor 12 could be designed more simply, which likewise means a cost saving. In other words, a smaller Δp leads to a more advantageous form of the gas compressor 12. This results in the form shown according to FIG. 2. This consists in lowering the pressure p1 of the absorbent 10 or 10′ gradually, stepwise via one or more cascades to the pressure p2. These intermediate pressures, which each require a separate pressure chamber 18 or 118, 218, 318 and 418, lie between p1 and p2, and these intermediate pressures are denoted p11, p12, p13 and p14. The pressure drop between the respective pressure chambers 18 or 118 or 218 etc. is advantageously approximately 5 bar, so that a portion of the products 6 or also of the starting materials 4 is discharged from the absorbent 10′ in each pressure chamber 18, 118, 218, etc. Complete discharge of the absorbent 10′ does not take place in any of the mentioned pressure chambers 18, merely partial discharge. Only in the last pressure chamber, according to this nomenclature 418, is the absorbent 10′ lowered to the final pressure and relief pressure p2 and discharged completely there.

The process steps already described of condensing the products 6 in a condensation device 20 and feeding the uncondensed starting materials 4 into the respective liquid-jet gas compressor takes place in an analogous manner, as already described in FIG. 1. In principle, correspondingly more individual components are required in this cascaded arrangement than in the representation according to FIG. 1, but, on the other hand, the liquid-jet gas compressor 12 and the pump 24 can be configured significantly more advantageously than the gas compressor 12 and the pump 24 according to FIG. 1. Which method is ultimately the more economical depends to a very large extent on the process management and on the individual costs of the individual process components.

### LIST OF REFERENCE SIGNS

2 reactor system
4 starting materials
6 products
8 reaction chamber
10 absorbent
10′ laden absorbent
10″ absorbent
12 liquid-jet compressor
14 reactor bundle
16 valves
18 pressure chamber 118, 218, 318, 418
20 condensation means
22 cooling device
24 pump
V_{F} volume flow rate absorbent inlet
V_{G} volume flow rate gases
V volume flow rate mixture
p1 pressure
p2 pressure
p3 pressure
p4 pressure
p11 pressure

1. A method for operating a reactor system for carrying out equilibrium-limited reactions, comprising:
converting starting materials into a product in a reaction chamber under a pressure p1, wherein an absorbent is laden with the product and thereby also absorbs starting materials,
a) discharging the laden absorbent from the reaction chamber, and
b) lowering the pressure of the absorbent to a pressure p2, wherein the pressure p2 is lower than the pressure p1, and wherein the product and starting materials are discharged in a gaseous state from the liquid absorbent and
c) the gaseous products are separated from the gaseous starting materials by condensation,
d) while a pressure p3 is applied to the liquid absorbent, the pressure p3 being higher than the pressure p1, and
e) the liquid absorbent is conducted under the pressure p3 into a liquid-jet gas compressor, in which the gaseous starting materials separated from the products are aspirated and dissolved in the liquid absorbent and
f) are then introduced into the reaction chamber under the pressure p4, which is lower than the pressure p3.

2. The method according to claim 1,
wherein the pressure of the absorbent is lowered from the pressure p1 to the pressure p2 over a pressure stage p11 and method steps c) and e) are also carried out analogously for the pressure p11.

3. The method according to claim 1,
wherein the pressure p1 lies between 20 bar and 250 bar.

4. The method according to claim 1,
wherein the pressure p2 lies between 1 bar and 50 bar.

5. The method according to claim 2,
wherein the pressure p11 is between 3 bar and 10 bar lower than the pressure p1.
